(19) United States
(12) Patent Application Publication
(10) Pub. No.: US 2010/0028319 A1
(43) Pub. Date: Feb. 4, 2010

(54) COMPOSITION FOR PREVENTION OR TREATMENT OF DIABETES

(75) Inventors: Hirokazu Sakamoto, Hyogo (JP); Hideyuki Kishida, Hyogo (JP); Kazunori Hosoe, Hyogo (JP)

Correspondence Address: HAMRE, SCHUMANN, MUELLER & LARSON, P.C., P.O. BOX 2902, MINNEAPOLIS, MN, 55402-0902, US

(73) Assignee: KANEKA CORPORATION, Osaka-shi, Osaka (JP)

(21) Appl. No.: 12/525,446

(22) PCT Filed: Jan. 31, 2008
(86) PCT No.: PCT/JP2008/051474

§ 371 Date: Sep. 24, 2009

(30) Foreign Application Priority Data
Feb. 2, 2007 (JP) 2007-024920

(51) Int. Cl. A61K 38/43; C07H 19/20; A61P 7/12

(52) U.S. Cl. 424/94.1; 536/26.7

(57) ABSTRACT

The present invention aims to provide a composition which has an extremely broad range of applications, has an excellent safety, and can be used for preventing, ameliorating, or therapeutically-treating diabetes.

The present invention provides an agent for preventing, ameliorating, or therapeutically-treating diabetes or a diabetic complication, a glycemic control agent, and an insulin secretion-promoting agent, each of which contains reduced coenzyme A as an active ingredient. The present invention also provides a method for preventing, ameliorating, or therapeutically-treating diabetes or a diabetic complication, which includes administration of the aforementioned agent.

### TECHNICAL FIELD

The present invention relates to an agent for preventing, ameliorating, or therapeutically-treating diabetes and diabetic complications, a glycemic control agent, an insulin secretion-promoting agent, a composition used in combination with a pharmaceutical for therapeutically-treating diabetes or a diabetic complication, a diabetic diet, and various kinds of compositions containing them.

### BACKGROUND ART

Diabetes is caused by disorders of metabolism, mainly glucose metabolism, owing to lack of insulin secretion, reduction of sensitivity of the target cells for insulin, or the like. The prominent feature of diabetes is induction of hyperglycemia. A long-lasting hyperglycemia causes vascular disorders, and the vascular disorders become a key factor to develop serious complications in various organs or nerves, such as retinopathy, nephropathy, and neurological disorders. Therefore, in therapeutic treatments of diabetes, it is of the utmost importance to control and maintain the blood glucose level within a normal range, and means for this purpose have long been researched.

For glycemic control in type-I diabetic patients, insulin injection is profoundly effective since hyposecretion of insulin causes the symptom. On the other hand, in many cases, insulin injection does not work well on type-II diabetic patients who have a low sensitivity to insulin. For therapeutic treatment of the type-II diabetic patients, diet control is firstly performed to limit excessive calorie intake from diet, and further an exercise therapy is employed to accelerate calorie consumption in the body. However, the diet control is in many cases difficult to continue. For the patients who do not show any notable progress by the diet control and the exercise therapy, administration of a diabetic drug (sulfonylurea drug, biguanide drug, etc.) is tried; however, such diabetic drugs have serious problems. Namely, it is known that the sulfonylurea drug (SU drug), which has a long history of use, with its second-generation drug used since 1970s, may excessively lower the blood glucose level to cause hypoglycemia. Moreover, since the sulfonylurea drug increases hunger, the patients sometimes cannot follow a controlled diet, often resulting in promotion of obesity as well as failure of the glycemic control. For use of a SU drug having the foregoing problems, strict control by a doctor is necessary. As for a biguanide drug (BG drug), which has been used since 1970s, a large number of cases of lactic acidosis as a side effect were reported soon after it was first introduced. Although an increasing attention has been again paid to the method for use and effects of the drug in these days, the drug must be used under strict supervision by a doctor. In such circumstances, as a result of an attempt to develop new oral antidiabetics, an α-glucosidase inhibitor and an insulin action enhancer were developed. However, as there are few reports using those new drugs, it is necessary to wait for future investigations before deciding whether or not they are safe and effective. In fact, development of Troglitazone, an insulin action enhancer, was discontinued due to its side effect which causes liver damage. As mentioned earlier, there are very few safe oral antidiabetics which patients can use without anxiety, and thus it may be not too much to say that diet control forms a major part of a practical therapeutic treatment. However, strictly carrying out the diet control is actually difficult in many cases. Although a large number of low calorie foods for diabetic patients have been developed for easier prosecution of the diet control, their contribution to therapeutic treatment of diabetes is merely partial. Furthermore, as there is no therapeutic agent for complications that characteristically occur with diabetes, strict control of the blood glucose level is also required to prevent the complications in the present circumstances. Accordingly, for treatment of diabetes, there has been a demand for a safe and effective drug or an effective food to be safely taken without anxiety during a diet control period.

Coenzyme A (also referred to as CoA) is an active substance that is present in high amounts in living bodies. Coenzyme A comprises pantothenic acid, adenine, ribose, cysteamine, and phosphoric acid. It is a coenzyme for acetylation in vivo, and plays a significantly important role in the metabolism of carbohydrates, lipids, and proteins. For example, coenzyme A is essential for the process of tricarboxylic acid cycle, accumulation of liver glycogen, synthesis of acetylcholine, control of cholesterol level, control of plasma lipid concentration, and synthesis of steroid. Coenzyme A in vivo is mostly present in the reduced form as the same with reduced coenzyme A used in the present invention.

It is reported that intake of coenzyme A greatly alleviates the symptoms like loss of appetite and hypokinesia of patients, and is effective for arteriosclerosis, chronic arteritis, cardiac infarction, myocarditis, hyperlipemia (Patent Document 1), various hepatic diseases such as hepatitis, cirrhosis, hepatic coma, jaundice, and fatty liver (Non-Patent Document 1 and Non-Patent Document 2), wound healing (Patent Document 2), periodontal disease (Patent Document 3), and other diseases. Further, in the field of beauty, it is reported that coenzyme A has skin anti-aging effects such as UV ray protection, prevention of spots and wrinkles, skin brightening effect, and moisturizing effect (Patent Documents 4 and 5).

Patent Document 1 JP-A 2001-64184

Patent Document 2 JP-A S63-152309

Patent Document 3 JP-A H08-245352

Patent Document 4 JP-A H02-49729

Patent Document 5 JP-A S50-31051

Non-Patent Document 1 Naika Hokan (Japanese archives of internal medicine) 15(3), 67-75, 1968 Non-Patent Document 2 Sanfujinka no Jissai (Practice of obstetrics and gynecology) 18(6), 545-557, 1969

### SUMMARY OF THE INVENTION

As mentioned earlier, problematically, application of conventional methods for therapeutically-treating diabetes does not provide desired effects. Moreover, in a drug therapy, a side effect may be caused in addition to the efficacy of the drug.

The present invention aims to provide a composition which is very safe, promotes insulin secretion, and can be used for preventing, ameliorating, or therapeutically-treating diabetes. Furthermore, the present invention aims to provide a method for treating diabetes or diabetic complications with use of the composition of the present invention.

The present inventors carried out intensive examinations to achieve the aims. As a result, they have found that, surprisingly, reduced coenzyme A causes increase in the ATP level in pancreatic β cells so as to activate the cells, and furthermore, reduced coenzyme A significantly promotes insulin secretion in response to glucose load. Namely, the present invention has one or a plurality of aspects as follows:

(1) an insulin secretion-promoting agent, containing reduced coenzyme A as an active ingredient;

(2) a glycemic control agent, containing the agent according to the above (1);

(3) an agent for preventing, ameliorating, or therapeutically-treating diabetes or a diabetic complication, containing the agent according to the above (1);

(4) an agent for preventing, ameliorating, or therapeutically-treating diabetes or a diabetic complication, containing reduced coenzyme A and a pharmaceutical ingredient used for therapeutically-treating diabetes and/or a diabetic complication;

(5) the agent according to any one of the above (1) to (4), further containing oxidized coenzyme A;

(6) the agent according to any one of the above (1) to (5), wherein the agent is in an orally administrable form;

(7) a diabetic diet, containing the agent according to any one of the above (1) to (5);

(8) a food, containing the agent according to any one of the above (1) to (5);

(9) a pharmaceutical, containing the agent according to any one of the above (1) to (5);

(10) a quasi-drug, containing the agent according to any one of the above (1) to (5);

(11) a nutrition supplement, containing the agent according to any one of the above (1) to (5);

(12) a veterinary pharmaceutical, containing the agent according to any one of the above (1) to (5);

(13) a pet food, a feed or a feed organism, containing the agent according to any one of the above (1) to (5);

(14) a method for treating diabetes or a diabetic complication, including administering reduced coenzyme A,

(15) the method according to the above (14), wherein the reduced coenzyme A is administered orally;

(16) the method according to the above (14), wherein a subject of administration is a diabetic patient or a diabetic pre-patient;

(17) the method according to the above (14), further including administering a pharmaceutical ingredient used for therapeutically-treating diabetes and/or a diabetic complication;

(18) use of reduced coenzyme A for preventing, ameliorating, or therapeutically-treating diabetes; and

(19) use of reduced coenzyme A for manufacturing a medicament for preventing, ameliorating, or therapeutically-treating diabetes.

The above (2) and (3) may be paraphrased as follows:

(2′) a glycemic control agent, containing reduced coenzyme A as an active ingredient; and

(3′) an agent for preventing, ameliorating, or therapeutically-treating diabetes or a diabetic complication, containing reduced coenzyme A as an active ingredient.

The composition containing reduced coenzyme A as an active ingredient according to the present invention can cause increase in the ATP level in pancreatic β cells so as to activate the cells. Moreover, the composition can promote insulin secretion in response to glucose load. Furthermore, the composition has no side effects problematically observed in conventional drugs, and thus it can be used safely and can be applied as an agent for preventing, ameliorating, or therapeutically-treating diabetes. Additionally, it is possible to provide a glycemic control method using the composition of the present invention and a method for preventing diabetes or a diabetic complication using the composition of the present invention.

### BRIEF DESCRIPTION OF THE DRAWINGS

FIG. 1 is a graph showing influences of reduced coenzyme A on the intracellular ATP level in HIT-T15 cells.

FIG. 2 is a graph showing influences of reduced coenzyme A-related substances on the intracellular ATP level in HIT-T15 cells.

FIG. 3 is a graph showing influences of reduced coenzyme A on glucose-responsive insulin secretion in HIT-T15 cells.

### BEST MODE FOR CARRYING OUT THE INVENTION

In accordance with the present invention, reduced coenzyme A is a compound represented by the following formula:

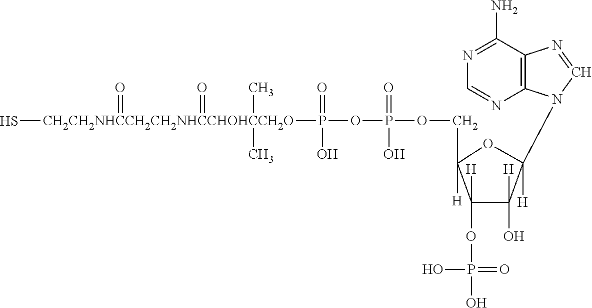

One aspect of the present invention is an agent for preventing, ameliorating, or therapeutically-treating diabetes and diabetic complications, a glycemic control agent, or an insulin secretion-promoting agent, each of which contains reduced coenzyme A as an active ingredient. Another aspect of the present invention is a diabetic diet, a food, a pharmaceutical, a quasi-drug, a nutrition supplement, a veterinary pharmaceutical, a pet food, a feed, or a feed organism, each of which contains the foregoing agent. Still another aspect of the present invention is a method for treating diabetes or a diabetic complication using the composition of the present invention.

As will be demonstrated by Examples, reduced coenzyme A used in the present invention caused increase in the intracellular ATP level in pancreatic β cells and activated the cells. Direct activation of cells by increasing ATP, which supplies energy to cells, improves functions of the cells, leading to efficient exertion of the cell functions. This is very effective for maintaining or promoting health on a daily basis, and is further very effective for ameliorating abnormalities of various cell functions caused by various kinds of diseases, aging and the like.

Moreover, as will be evident from Examples, reduced coenzyme A used in present invention significantly promoted insulin secretion in response to glucose load. This result indicates that reduced coenzyme A used in the present invention can moderate elevation of blood glucose level after meals. Further, reduced coenzyme A used in the present invention can improve insulin secretion in diabetic patients and suspected diabetic patients, thereby restoring their blood glucose level to normal. Therefore, the reduced coenzyme A is effective for preventing development of diabetes, or ameliorating or therapeutically-treating diabetes, and preventing, ameliorating, or therapeutically-treating diabetic complications. It is to be noted that “preventing” used herein means reduction of the risks of contraction or development of the foregoing diseases.

The composition containing reduced coenzyme A according to the present invention includes reduced coenzyme A as an active ingredient. In addition to the reduced coenzyme A, the composition according to the present invention may further include oxidized coenzyme A represented by the following formula:

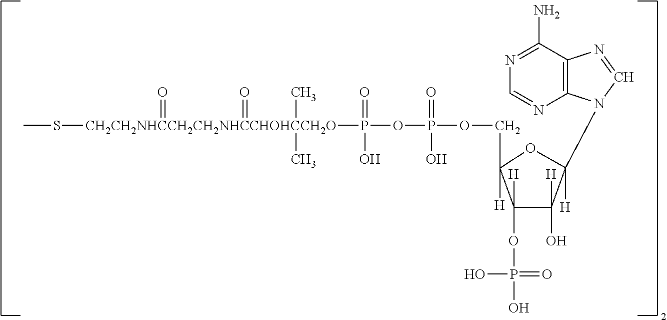

and/or a degradation product of these coenzyme A.

The composition containing reduced coenzyme A according to the present invention may be applied to animals as well as human beings. The human subject to be administered is not particularly limited; however, the composition according to the present invention is especially effective for patients with central or peripheral nervous disorders and pre-patients thereof. Examples of the pre-patients of central or peripheral nervous disorders include people who may hereditarily or epidemiologically have higher risks of developing such disorders and people who may have higher risks of developing such disorders on the grounds of the results of medical checkups or tests, as compared with healthy people.

The composition containing reduced coenzyme A according to the present invention may further contain various additives that are acceptable pharmaceutically or under the Food Sanitation Law, in addition to the reduced coenzyme A. Further, when used as measures against various diseases, the composition containing reduced coenzyme A of the present invention may be used in combination with pharmaceuticals or active ingredients against the diseases.

Examples of the pharmaceuticals that may be used in combination with the composition of the present invention include those pharmaceuticals generally used as diabetic drugs, such as insulin preparations, sulfonylurea drugs, α-glucosidase inhibitors, biguanide drugs, insulin resistance-improving agents, insulin action enhancers, and fast-acting postprandial hypoglycemic agents.

The composition of the present invention may be used in combination with various pharmaceuticals used for therapeutically-treating diabetic complications, during the treatment thereof. Examples of the diabetic complications include arteriosclerosis, ischemic heart diseases (e.g. myocardial infarction, angina pectoris), cerebral infarction, infections due to immune compromise, diabetic nervous disorders (e.g. facial paralysis, sudden deafness, nausea, vomiting, diarrhea, constipation, urination disorder, erectile dysfunction, dystrophy, Charley horse, numbness or pain in hands and feet, hypesthesia, dyshidrosis, cutaneous ulcer and gangrene), diabetic retinopathy, and diabetic nephropathy.

The foregoing pharmaceuticals and the reduced coenzyme A may be taken separately or in combination as one composition.

The nutrition supplement according to the present invention refers to a product to maintain health or to ameliorate various diseases by oral intake, and examples thereof include oral supplements, foods for specified health uses, health foods, dietary supplements, and other products except for pharmaceuticals.

The composition according to the present invention may contain health food ingredients, dietary supplement ingredients, vitamins and other additives. Such additives is not particularly limited, and examples thereof include vehicles, disintegrating agents, lubricants, binders, coating agents, colorants, anti-aggregation agents, absorption enhancers, solubilizing agents, stabilizers, health food ingredients, dietary supplement ingredients, vitamins, flavors, sweeteners, antiseptics, preservatives, and anti-oxidants.

The vehicles are not particularly limited, and examples thereof include sucrose, lactose, glucose, cornstarch, mannitol, crystalline cellulose, calcium phosphate and calcium sulfate.

The disintegrating agents are not particularly limited, and examples thereof include starches, agar, calcium citrate, calcium carbonate, sodium bicarbonate, dextrin, crystalline cellulose, carboxymethyl cellulose and tragacanths.

The lubricants are not particularly limited, and examples thereof include magnesium stearate, sodium stearate, stearic acid, calcium stearate, magnesium oleate, oleic acid, potassium oleate, caprylic acid, sodium stearyl fumarate, and magnesium palmitate, and compositions containing those lubricants.

The binders are not particularly limited, and examples thereof include starches and derivatives thereof (e.g. pregelatinized starch, dextrin), celluloses and derivatives thereof (e.g. ethyl cellulose, sodium carboxyl methyl cellulose, hydroxy propyl methyl cellulose), gum arabic, tragacanths, gelatin, saccharides (e.g. glucose, sucrose), ethanol, and polyvinyl alcohol.

The coating agents are not particularly limited, and examples thereof include cellulose derivatives (e.g. hydroxypropyl cellulose, cellulose acetate phthalate, hydroxypropyl methylcellulose phthalate), shellac, polyvinyl pyrrolidone, polyvinyl pyridines (e.g. poly-2-vinyl pyridine, poly-2-vinyl-5-ethyl pyridine), polyvinylacetyl diethylamino acetate, polyvinyl alcohol phthalate, and copolymers of methacrylate and methacrylic acid.

The colorants are not particularly limited, and examples thereof include those colorants which are allowed to be added in pharmaceuticals and foods, such as Blue No. 1, Yellow No. 4, Green No. 3, Red No. 5, a lake pigment, titanium dioxide, red cabbage colorant, monascus pigment, purple sweet potato pigment, Gardenia pigment, and cochineal pigment.

The absorption enhancers are not particularly limited, and examples thereof include surfactants such as higher alcohols, higher fatty acids, and glycerin fatty acid ester.

The solubilizing agents are not particularly limited, and examples thereof include adipic acid, L-arginine, sodium benzoate, benzyl benzoate, esterified corn oil, ethanol, magnesium chloride, hydrochloric acid, olive oil, carmellose sodium, dried sodium carbonate, dilute hydrochloric acid, citric acid, sodium citrate, glycine, glycerin, glycerin fatty acid ester, geraniol, sesame oil, cellulose acetate phthalate, sodium salicylate, magnesium oxide, α-cyclodextrin, β-cyclodextrin, dibutyl hydroxy toluene, tartaric acid, sucrose fatty acid ester, sodium hydroxide, sorbitan sesquioleate, sorbitan fatty acid ester, D-sorbitol, D-sorbitol syrup, soybean oil, soybean lecithin, sodium bicarbonate, sodium carbonate, medium-chain fatty acid triglyceride, triacetin, sorbitan trioleate, nicotinamide, lactic acid, concentrated glycerin, cupronickel, hydroxypropyl methylcellulose, castor oil, glacial acetic acid, glucose, propylene glycol, propylene glycol fatty acid ester, povidone, polyoxyethylene hydrogenated castor oil, polyoxyethylene (160) polyoxypropylene (30) glycol, polysorbate, polyvinyl alcohol, macrogol, D-mannitol, isopropyl myristate, dehydrated ethanol, anhydrous citric acid, sorbitan monooleate, lauryl macrogol, lidocaine, phosphoric acid, sodium hydrogen phosphate, and potassium dihydrogen phosphate.

The stabilizers are not particularly limited, and examples thereof include benzoic acid, sodium benzoate, and ethyl p-hydroxybenzoate.

The health food ingredients are not particularly limited, and examples thereof include Kampo (Chinese) medicines, tea leaves, herbs, propolis, Ginkgo biloba leaves, Aojiru (green drink), and extracts thereof.

Examples of the Kampo (Chinese) medicines include Irei-to, Unkei-to, Unsei-in, Ogi-kenchu-to, Oren-gedoku-to, Oren-to, Kakkon-to, Kami-kihi-to, Kami-shoyo-san, Kam-baku-taiso-to, Kikyo-to, Kihi-to, Kumi-binro-to, Keigai-rengyo-to, Keishi-ka-shakuyaku-daio-to, Keihi-ka-shakuyaku-to, Keihi-ka-ryukotsu-borei-to, Keishi-to, Keishi-ninjin-to, Keishi-bukuryo-gan, Keihi-to, Koso-san, Goko-to, Goshaku-san, Gosha-jinki-gan, Gorin-san, Saikan-to, Saiko-ka-ryukotsu-borei-to, Saiko-keishi-kankyo-to, Saiko-keishi-to, Saiko-seikan-to, Saiboku-to, Sairei-to, Sansonin-to, Jiin-koka-to, Shigyaku-san, Shikunshi-to, Shimotsu-to, Sha-kanzo-to, Shakuyaku-kanzo-to, Juzen-taiho-to, Jumi-haidoku-to, Sho-kenchu-to, Sho-saiko-to, Sho-seiryu-to, Shofu-san, Shin-i-seihai-to, Shimpi-to, Shimbu-to, Seijo-bofu-to, Seisho-ekki-to, Seishin-renshi-in, Seihai-to, Sokei-kakketsu-to, Daio-kanzo-to, Daio-botampi-to, Dai-kenchu-to, Dai-saiko-to, Dai-saiko-to-kyo-daio, Dai-joki-to, Dai-bofu-to, Ji-daboku-ippo, Choi-joki-to, Choto-san, Choyo-to, Chorei-to, Chorei-to-go-shimotsu-to, Tsu-do-san, Tokaku-joki-to, Toki-inshi, Toki-kenchu-to, Toki-shakuyaku-san, Toki-to, Nichin-to, Nyoshin-san, Ninjin-to, Ninjin-yoei-to, Haino-san-kyu-to, Bakumondo-to, Hachimi-jio-gan, Hange-koboku-to, Hange-shashin-to, Byakko-ka-ninjin-to, Bukuryo-in, Bukuryo-in-go-hange-koboku-to, Heii-san, Boi-ogi-to, Bofu-tsusho-san, Hochu-ekki-to, Mao-to, Mao-bushi-saishin-to, Ma-kyo-kan-seki-to, Mashinin-gan, Moku-boi-to, Yoku-kan-san, Yoku-kan-san-ka-chimpi-hange, Rikkunshi-to, Rikko-san, Ryutan-shakan-to, Ryo-kan-kyo-mi-shin-ge-nin-to, and Rokumi-gan.

Examples of the tea leaves include green tea, Genmaicha (Japanese tea mixed with roasted brown rice), Matcha (powdered green tea), Sencha (green tea of middle grade), Hojicha (toasted tea), roasted tea, jasmine tea, oolong tea, black tea, black colored tea, flower scented tea, blue tea, and white tea.

Examples of the herbs include Italian parsley, elecampane, olive, oregano, cardoon, chamomile, curry plant, catnip, caraway, Christmas rose, crimson clover, cornflower, common mallow, salad burnet, santolina, cinnamon, jasmine, stevia, sage, common lime, scented geranium, St John's wort, soapwort, Solomon's seal, thyme, tansy, chervil, chive, nasturtium, jujube, basil, honeysuckle, hyssop, flax, fennel, foxglove, black hollyhock, French marigold, betony, heliotrope, bergamot, hemp agrimony, common rue, pot marigold, borage, white horehound, myrtle, mullein, marjoram, mint, yarrow, lavender, Lady's bedstraw, lemongrass, lemon verbena, lemon balm, rose, rosemary, rocket, wild strawberry, wild pansy, forget-me-not, and other herbs.

The dietary supplement ingredients are not particularly limited, and examples thereof include amino acids, metal ions, proteins, saccharides, fatty acids, yeast extracts, vegetable extracts, fish meat extracts, fruits, fruit extracts, N-acetyl glucosamine, S-adenosyl methionine, tetrahydrobiopterin, and oxidized coenzyme Q10.

The vitamins are not particularly limited, and examples thereof include vitamin A, vitamin B, vitamin C, vitamin D, vitamin E, vitamin K, and derivatives thereof.

Examples of the flavors include: single flavors such as menthol, carvone, anethole, cineol, methyl salicylate, cinnamic aldehyde, eugenol, 3,1-menthoxypropane-1,2-diol, thymol, linalool, lynalyl acetate, limonene, menthone, menthyl acetate, N-substituted-p-menthane-3-carboxamide, pinene, octyl aldehyde, citral, pulegone, carvyl acetate, anisaldehyde, ethyl acetate, ethyl butyrate, allyl cyclohexane propionate, methyl anthranilate, ethyl methyl ethynyl glycidate, vanillin, undecalactone, hexanal, ethyl alcohol, propyl alcohol, butanol, isoamyl alcohol, hexenol, dimethyl sulfide, cyclotene, furfural, trimethylpyrazon, ethyllactate, and ethyl thio acetate;

natural flavors such as peppermint oil, spearmint oil, anise oil, eucalyptus oil, wintergreen oil, cassia oil, clove oil, thyme oil, sage oil, lemon oil, orange oil, mentha oil, cardamom oil, coriander oil, mandarin oil, lime oil, lavender oil, rosemary oil, laurel oil, chamomile oil, caraway oil, marjoram oil, bay oil, lemon grass oil, origanum oil, pine needle oil, neroli oil, rose oil, jasmine oil, iris concrete, peppermint absolute, rose absolute and orange flower; and

compound flavors such as strawberry flavor, apple flavor, banana flavor, pineapple flavor, grape flavor, mango flavor, butter flavor, milk flavor, fruit mix flavor, and tropical fruit flavor.

Examples of the sweeteners include saccharin sodium, aspartame, stevioside, stevia extract, p-methoxy cinnamic aldehyde, neohesperidyl dihydrochalcone, and perillartine.

Examples of the antiseptics include p-oxybenzoic acid esters such as butylparaben and ethylparaben, sodium benzoate, aminoethylsulfonic acid, benzoic acid, sodium benzoate, ethanol, disodium edetate, agar, dL-camphor, citric acid, sodium citrate, salicylic acid, sodium salicylate, phenyl salicylate, dibutyl hydroxy toluene, sorbic acid, potassium sorbate, nitrogen, dehydroacetic acid, sodium dehydroacetate, 2-naphthol, sucrose, honey, isobutyl p-hydroxybenzoate, isopropyl p-hydroxybenzoate, ethyl p-hydroxybenzoate, butyl p-hydroxybenzoate, propyl p-hydroxybenzoate, methyl p-hydroxybenzoate, L-menthol, and eucalyptus oil.

Examples of the preservatives include benzoic acid, sodium benzoate, ethanol, disodium edetate, dried sodium sulfite, citric acid, glycerin, salicylic acid, sodium salicylate, dibutyl hydroxy toluene, D-sorbitol, sorbic acid, potassium sorbate, sodium dehydroacetate, isobutyl p-hydroxybenzoate, isopropyl p-hydroxybenzoate, ethyl p-hydroxybenzoate, butyl p-hydroxybenzoate, propyl p-hydroxybenzoate, methyl p-hydroxybenzoate, propylene glycol, and phosphoric acid.

Examples of the anti-oxidants include citric acid, citric acid derivatives, vitamin C, vitamin C derivatives, licopene, vitamin A, carotenoids, vitamin B, vitamin B derivatives, flavonoides, polyphenols, selenium, sodium thiosulfate, vitamin E, vitamin E derivatives, α-lipoic acid, α-lipoic acid derivatives, pycnogenol, flavangenol, superoxide dismutase (SOD), glutathione peroxidase, glutathione-S-transferase, glutathione reductase, catalase, ascorbic acid peroxidase, reduced coenzyme Q10, glutathione, and mixtures thereof.

The administration form of the composition containing reduced coenzyme A according to the present invention may be either liquid or solid.

Examples of the administration form of the composition of the present invention as a pharmaceutical include oral agents (liquid formulations such as extracts, elixirs, syrups, tinctures, and lemonades; and solid formulations such as capsules, granules, pills, powders, and tablets), injections, nasal drops, eye drops, suppositories and sprays, and cutaneous administration forms such as ointments and adhesive skin patches.

When administered as a food, the composition containing reduced coenzyme A according to the present invention may be taken as it is or as a food or drink containing the composition. It is also possible to add reduced coenzyme A used in the present invention in foods for special dietary use such as prescription foods for diabetic patients.

When administered as a nutrition supplement, the composition containing reduced coenzyme A according to the present invention may be taken as it is or as a food or drink containing the composition. Further, in the same manner as above, the composition containing reduced coenzyme A according to the present invention can be used as health supplements, foods for specified health use, and foods with nutrient function claims.

Examples of the administration form of the composition of the present invention as a veterinary pharmaceutical include oral agents (liquid formulations such as extracts, elixirs, syrups, tinctures, and lemonades; and solid formulations such as capsules, granules, pills, powders, and tablets), injections, nasal drops, eye drops and suppositories, and cutaneous administration forms such as ointments and adhesive skin patches.

When administered as a pet food, a feed or a feed organism, the composition containing reduced coenzyme A according to the present invention may be fed as it is or as a food or drink containing the composition. Further, in the same manner as above, the composition can be used as health foods for animals and dietary supplements for animals.

The composition of the present invention has glycemic control effects based on its insulin secretion-promoting action. Accordingly, the composition of the present invention is effective for preventing development of diabetes, therapeutically-treating diabetes, preventing diabetic complications, and so forth. The composition of the present invention is thus usable in a glycemic control method, a method for treating, namely, for preventing, ameliorating or therapeutically-treating diabetes or a diabetic complication, and the like.

### EXAMPLES

Example 1 shows effects of reduced coenzyme A on ATP production in pancreatic β cell lines (hereinafter referred to as HIT-T15 cells). Example 2 shows influences of reduced coenzyme A on glucose-responsive insulin secretion in HIT-T15 cells.

### Example 1

### Effects of Reduced Coenzyme A and Reduced Coenzyme A-Related Substances on ATP Production in HIT-T15 Cells

Ham'F12 medium (GIBCO) containing 200 units of penicillin (GIBCO), 0.1 g/ml streptomycin (GIBCO), and 10% fetal bovine serum (GIBCO) is normally used as a growth medium for cultivating HIT-T15 cells.

A growth medium containing a fetal bovine serum at a concentration of 0.5% was placed in each well of a 96-well microplate, and HIT-T15 cells were seeded in the wells at a density of 1×10⁴ cells/well. Next day, the growth medium of each well was replaced with a growth medium containing 0.5% fetal bovine serum, in which reduced coenzyme A was further added to a final concentration of 6.25, 12.5, 25, 50 or 100 μg/ml. Twenty-four hours later, the medium of each well was removed and replaced with a growth medium (containing 10% fetal bovine serum) in which reduced coenzyme A was added at the same concentration as mentioned above, and cultivation was continued for another 24 hours. After washing three times with PBS, the intracellular ATP level was measured using ATPLite (PerkinElmer).

A growth medium containing a fetal bovine serum at a concentration of 0.5% was placed in each well of a 96-well microplate, and the cells were seeded in the wells at a density of 1×10⁴ cells/well. Next day, the growth medium of each well was replaced with a growth medium containing 0.5% fetal bovine serum, in which reduced coenzyme A or three kinds of coenzyme A precursors (pantothenic aid, pantethine, cystein) or oxidized coenzyme A was added. The coenzyme A precursors and the oxidized coenzyme A were added at the same molar concentrations as that of the reduced coenzyme A. Twenty-four hours later, the medium of each well was removed and replaced with a growth medium (containing 10% fetal bovine serum) in which reduced coenzyme A, the coenzyme A precursor, or oxidized coenzyme A was added at the same concentration as mentioned above, and cultivation was continued for another 24 hours. After washing three times with PBS, the intracellular ATP level was measured using ATPLite (PerkinElmer).

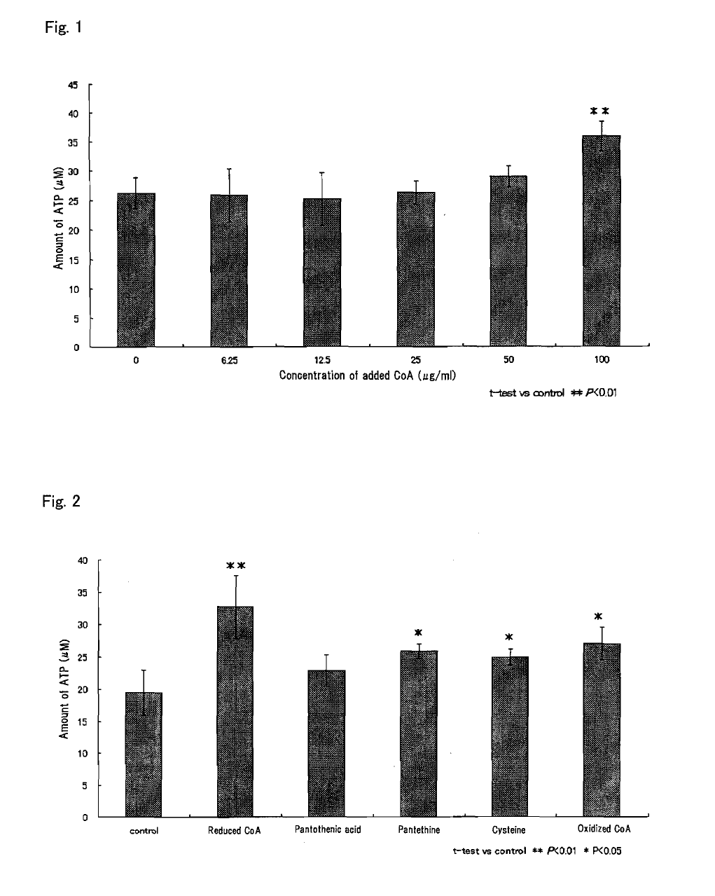

FIGS. 1 and 2 show the evaluation results. As is evident from the results, addition of reduced coenzyme A significantly increased the intracellular ATP level in HIT-T15 cells. Further, addition of reduced coenzyme A promoted ATP production significantly in comparison to additions of the precursors of reduced coenzyme A, namely, of pantothenic acid, of pantethine and of cystein, and addition of a mixture of the three precursors. Furthermore, addition of reduced coenzyme A promoted ATP production significantly in comparison to addition of oxidized coenzyme A.

### Example 2

### Influence of Reduced Coenzyme A on Glucose-Responsive Insulin Secretion in HIT-T15 Cells

HIT-T15 cells were seeded in the wells of a 96-well plate at a density of 50,000 cells/100 μl medium/well. An RPMI 1640 medium (GIBCO) containing 1% fetal bovine serum and 6 mM glucose was used at this time. Twenty-four hours later, the medium of each well was replaced with an RPMI 1640 medium (containing 1% fetal bovine serum and 6 mM glucose) in which reduced coenzyme A was added to a final concentration of 50 μg/ml. Another 24 hours later, the medium of each well was replaced with an RPMI 1640 medium (containing 10% fetal bovine serum and 6 mM glucose) in which reduced coenzyme A was added to a final concentration of 50 μg/ml. Yet another 24 hours later, the wells were washed with Krebs-Ringer buffer containing 10 mM of HEPES (KRB-HEPES), and further added with 100 μl of KRB-HEPES, followed by culturing for one hour. Next, after removal of the KRB-HEPES, the wells were washed with the same buffer. To the wells was added 100 μl of KRB-HEPES containing 20 mM of glucose, and the supernatant liquid was collected at intervals of 30 minutes for storing frozen. At a later date, the insulin amount in the supernatant liquid samples was assayed using ELISA (Lebis).

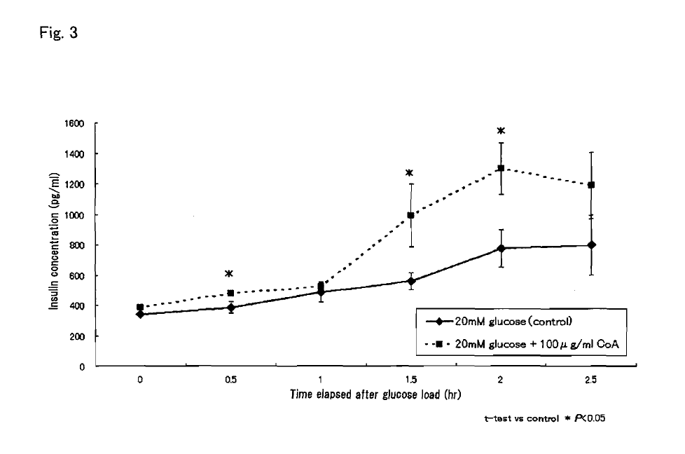

FIG. 3 shows the results. As is evident from the results, addition of reduced coenzyme A significantly promoted glucose-responsive insulin secretion in HIT-T15 cells.

### Example 3

### Preparation of Oral Formulation of Reduced Coenzyme A

The following ingredients were mixed and tableted in the usual manner to give tablets containing reduced coenzyme A.

Reduced coenzyme A: 10 g

Calcium carboxymethylcellulose (disintegrating agent): 0.4 g

Magnesium stearate (lubricant): 0.2 g

Crystalline cellulose (vehicle): 9.4 g

### INDUSTRIAL APPLICABILITY

Reduced coenzyme A used in the present invention can cause increase in the ATP level in pancreatic β cells so as to activate the cells. Moreover, the reduced coenzyme A can promote insulin secretion in response to glucose load. By blending reduced coenzyme A as an active ingredient, it is possible to produce a composition which causes no problems including side effects, has an extremely broad range of applications, has an excellent safety, promotes insulin secretion and thereby can control blood glucose level (for example, moderate elevation of blood glucose level after meals), and can be used for preventing, ameliorating, or therapeutically-treating diabetes. The composition can be favorably used, for example, in compositions for oral administration, compositions for oral cavity, foods, pharmaceuticals, quasi-drugs, nutrition supplements, veterinary pharmaceuticals, pet foods, feeds, and feed organisms. Moreover, the composition of the present invention can be used in combination with pharmaceuticals to be used for therapeutically-treating diabetes or diabetic complications. Additionally, it is possible to provide a glycemic control method using the composition of the present invention and a method for preventing diabetes or a diabetic complication with use of the composition of the present invention.

1. An insulin secretion-promoting agent, containing reduced coenzyme A as an active ingredient.

2. A glycemic control agent, containing the agent according to claim 1.

3. An agent for preventing, ameliorating, or therapeutically-treating diabetes or a diabetic complication, containing the agent according to claim 1.

4. An agent for preventing, ameliorating, or therapeutically-treating diabetes or a diabetic complication, containing reduced coenzyme A and a pharmaceutical ingredient used for therapeutically-treating diabetes and/or a diabetic complication.

5. The agent according to claim 1, further containing oxidized coenzyme A.

6. The agent according to claim 1,
wherein the agent is in an orally administrable form.

7. A diabetic diet, containing the agent according to claim 1.

8. A food, containing the agent according to claim 1.

9. A pharmaceutical, containing the agent according to claim 1.

10. A quasi-drug, containing the agent according to claim 1.

11. A nutrition supplement, containing the agent according to claim 1.

12. A veterinary pharmaceutical, containing the agent according to claim 1.

13. A pet food, a feed or a feed organism, containing the agent according to claim 1.

14. A method for treating diabetes or a diabetic complication, comprising administering reduced coenzyme A.

15. The method according to claim 14,
wherein the reduced coenzyme A is administered orally.

16. The method according to claim 14,
wherein a subject of administration is a diabetic patient or a diabetic pre-patient.

17. The method according to claim 14, further comprising administering a pharmaceutical ingredient used for therapeutically-treating diabetes and/or a diabetic complication.

18. Use of reduced coenzyme A for preventing, ameliorating, or therapeutically-treating diabetes.

19. Use of reduced coenzyme A for manufacturing a medicament for preventing, ameliorating, or therapeutically-treating diabetes.

20. The agent according to claim 4, further containing oxidized coenzyme A.

21. The agent according to claim 4,
wherein the agent is in an orally administrable form.

22. A diabetic diet, containing the agent according to claim 4.

23. A food, containing the agent according to claim 4.

24. A pharmaceutical, containing the agent according to claim 4.

25. A quasi-drug, containing the agent according to claim 4.

26. A nutrition supplement, containing the agent according to claim 4.

27. A veterinary pharmaceutical, containing the agent according to claim 4.

28. A pet food, a feed or a feed organism, containing the agent according to claim 4.
